SEQUENCE LISTING (4 sequences)

SEQ ID NO: 1 - DNA, 22 bp, Homo sapiens
uguaacagcaacuccaugugga

SEQ ID NO: 2 - DNA, 22 bp, Artificial Sequence
  gemcitabine modified miR-194 with gemcitabine at positions 6, 9, 12, 14 and 15
  misc_feature (6)..(6): N is Gemcitabine molecule
  misc_feature (9)..(9): N is Gemcitabine molecule
  misc_feature (12)..(12): N is Gemcitabine molecule
  misc_feature (14)..(15): N is Gemcitabine molecule
uguaanagnaanunnaugugga

SEQ ID NO: 3 - DNA, 22 bp, Artificial Sequence
  modified miR-194 with gemcitabine at positions 6, 9, 12, 14 and 15 and a fluorouracil at positions 1, 3, 13, 17 and 19
  misc_feature (1)..(1): N is Fluorouracil molecule
  misc_feature (3)..(3): N is Fluorouracil molecule
  misc_feature (6)..(6): N is Gemcitabine molecule
  misc_feature (9)..(9): N is Gemcitabine molecule
  misc_feature (12)..(12): N is Gemcitabine molecule
  misc_feature (13)..(13): N is Fluorouracil molecule
  misc_feature (14)..(15): N is Gemcitabine molecule
  misc_feature (17)..(17): N is Fluorouracil molecule
  misc_feature (19)..(19): N is Fluorouracil molecule
ngnaanagnaannnnangngga

SEQ ID NO: 4 - DNA, 22 bp, Artificial Sequence
  fluorouracil modified miR-194 with a fluorouracil molecule at positions 1, 3, 13, 17 and 19
  misc_feature (1)..(1): N is Fluorouracil molecule
  misc_feature (3)..(3): N is Fluorouracil molecule
  misc_feature (13)..(13): N is Fluorouracil molecule
  misc_feature (17)..(17): N is Fluorouracil molecule
  misc_feature (19)..(19): N is Fluorouracil molecule
ngnaacagcaacnccangngga

(19) United States
(12) Patent Application Publication
(10) Pub. No.: US 2022/0145304 A1
(43) Pub. Date: May 12, 2022

(54) MODIFIED MICRORNAS AND THEIR USE IN THE TREATMENT OF CANCER

(71) Applicant: The Research Foundation for The State University of New York, Albany, NY (US)

(72) Inventors: Jingfang JU, Albany, NY (US); Andrew FESLER, Albany, NY (US)

(73) Assignee: The Research Foundation for The State University of New York, Albany, NY (US)

(21) Appl. No.: 17/439,025

(22) PCT Filed: Mar. 13, 2020
(86) PCT No.: PCT/US2020/022519

§ 371 Date: Sep. 14, 2021

Related U.S. Application Data
(60) Provisional application No. 62/818,190, filed on Mar. 14, 2019.

(51) Int. Cl. C12N 15/113; A61K 31/7115; A61K 31/713; A61K 45/06; A61P 35/00; C12N 15/11

(52) U.S. Cl. CPC C12N 15/1135; A61K 31/7115; A61K 31/713; C07H 21/02; A61P 35/00; C12N 15/111; A61K 45/06

(57) ABSTRACT

The present disclosure provides modified microRNA nucleic acid compositions that have one or more cytosine and/or uracil bases replaced with gemcitabine or a 5-halouracil, respectively. More specifically, the present disclosure reveals that the replacement of cytosine nucleotides within a microRNA nucleotide sequence with a gemcitabine molecule increases the ability of the microRNA to inhibit cancer progression and tumorigenesis. In addition, the present disclosure reveals that the replacement of cytosine nucleotides within a microRNA nucleotide sequence with a gemcitabine molecule and replacement of uracil bases with 5-halouracil increases the ability of the microRNA to inhibit cancer development. As such, the present disclosure provides various modified nucleic acid (e.g., microRNA) compositions having gemcitabine molecules incorporated in their nucleic acid sequences and methods for using the same. The present disclosure further provides pharmaceutical compositions comprising the modified nucleic acid compositions, and methods for treating cancers using the same.

### CROSS REFERENCE TO RELATED APPLICATIONS

This application claims the benefit of priority from U.S. Provisional Application No. 62/818,190, filed Mar. 14, 2019, the entire contents of which are incorporated herein by reference.

### GOVERNMENT SUPPORT

This invention was made with government support under grant number CA 197098 awarded by the National Institutes of Health. The government has certain rights in the invention.

### INCORPORATION BY REFERENCE OF SEQUENCE LISTING

The Sequence Listing in the ASCII text file, named as 050_9017_PCT_SequenceListing.txt of 3 KB bytes, and submitted to the United States Patent and Trademark Office via EFS-Web, is incorporated herein by reference.

### FIELD OF THE DISCLOSURE

The present disclosure is generally directed to nucleic acid compositions that include 2′2′-difluoro 2′deoxycytidine (gemcitabine). More specifically, the present disclosure provides modified microRNA compositions that contain one or more gemcitabine molecules and methods for using the same. Furthermore, the instant application provides pharmaceutical compositions that include the inventive nucleic acid compositions and methods for treating cancer using the same.

### BACKGROUND

MicroRNAs (miRNAs, miRs) are a class of highly conserved, non-coding small ribonucleic acid (RNA) molecules that mediate translation in a cell or organism by negatively regulating the expression of their target genes and thus causing translational arrest, messenger RNA (mRNA) cleavage or a combination thereof. See Bartel D P. Cell. (2009) 136(2):215-33. By targeting multiple transcripts, miRNAs regulate a wide range of biological processes including apoptosis, differentiation and cell proliferation; thus, aberrant microRNA function can lead to cancer (see Ambros V. Nature. (2004) 431 pp. 350-355) and as such, miRNAs have recently been identified as biomarkers, oncogenes or tumor suppressors. See, e.g., Croce, C M. Nat Rev Genet. (2009) 10 pp. 704-714.

According to the World Health Organization, Cancer is a leading cause of death worldwide, accounting for 8.8 million deaths in 2015. Lung cancer is the leading cause of cancer death in both men and women in the United States, with only 18.6% of patients diagnosed with lung cancer surviving beyond 5 years. Surveillance, Epidemiology, and End Results Program. SEER Cancer Stat Facts: Lung and Bronchus Cancer. National Cancer Institute. Bethesda, Md. (2018). There are two primary categories of lung cancer: non-small cell lung cancer and small cell lung cancer. Non-small cell lung cancer is further delineated by type of cancer cells present in a tissue. As such, non-small cell lung cancer is broken down into following sub-classes of lung cancer: squamous cell carcinoma (also called epidermoid carcinoma), large cell carcinoma, adenocarcinoma (i.e., cancer that originates in cells lining alveoli), pleomorphic, carcinoid tumor and salivary gland carcinoma. Meanwhile, there are two main types of small cell lung cancer: small cell carcinoma and combined small cell carcinoma. SEER Cancer Stat Facts: Lung and Bronchus Cancer. National Cancer Institute. Bethesda, Md. (2018). The most common treatment for non-small cell lung cancers is gemcitabine (2′,2′-difluoro 2′deoxycytidine), taxol (e.g., paclitaxel), cisplatin (a DNA cross-linking agent), and combinations thereof. However, many types of antibody-based therapeutics are also used to treat non-small cell lung cancer (e.g., gefitinib, pembrolizumab, alectinib). Small cell lung cancer is commonly treated by methotrexate, doxorubicin hydrochloride, and topotecan based chemotherapeutic agents.

Breast cancer is the second most common cancer in women, with the most common type of breast cancer being ductal carcinoma. Ductal carcinoma begins in the cells of the ducts. In contrast, lobular carcinoma, which is often found in both breasts, originates in the lobes or lobules. Many chemotherapeutic agents are used to treat breast cancer including, but not limited to, cytotoxic drugs such as taxols (e.g., paclitaxel, docetaxel), doxorubicin hydrochloride, 5-FU, gemcitabine hydrochloride, methotrexate, and tamoxifen citrate. In addition many antibody-based therapeutic agents are administered to treat various types of breast cancer, such as trastuzumab, olaparib and pertuzumab.

Pancreatic cancer is a deadly cancer that is very difficult to treat. See Siegel, R L et al. Cancer J. Clin. (2015) 65 pp. 5-29. Unique aspects of pancreatic cancer include a very low 5 year survival rate of less than 7%, late presentation, early metastasis and a poor response to chemotherapy and radiation. See Maitra A and Hruban R H, Annu Rev. Pathol. (2008) 3 pp. 157-188. To date gemcitabine-based chemotherapy (2′,2′-difluoro 2′deoxycytidine) is the gold standard for the treatment of pancreatic cancer, however the effect of therapeutic intervention is limited due to drug resistance. Oettle, H et al. JAMA (2013) 310 pp. 1473-1481.

Bladder cancer is a highly prevalent form of cancer in men and women. In 2015, there were an estimated 708,444 people living with bladder cancer in the United States, with approximately 2.3% of men and woman being diagnosed with bladder cancer at some point in their lives. Noone A M, et al. (eds). SEER Cancer Statistics Review, 1975-2015, National Cancer Institute. Bethesda, Md. (2018). The primary types of bladder cancer are: transitional cell carcinoma; squamous cell carcinoma; and adenocarcinoma. Drugs approved for the treatment of bladder cancer include, for example, doxorubicin hydrochloride, cisplatin, gemcitabine hydrochloride and valrubicin. Certain antibodies are also approved to treat bladder cancer, including atezolizumab, avelumab, durvalumab, pembrolizumab, and nivolumab.

Ovarian cancer is present in approximately 225,000 women in the United States, with approximately 12/100,000 women being newly diagnosed with ovarian cancer each year. Noone A M, et al. (eds). SEER Cancer Statistics Review, 1975-2015, National Cancer Institute. Bethesda, Md. (2018). There are three primary forms of ovarian cancer. Namely, ovarian epithelial cancer, fallopian tube cancer, and primary peritoneal cancer, which form in the tissue covering the ovary, lining the fallopian tube or peritoneum, respectively. Many chemotherapeutic agents are used to treat ovarian cancers including, but not limited to, cytotoxic drugs such as taxols (e.g., paclitaxel), doxorubicin hydrochloride, toptecan hydrochloride, gemcitabine hydrochloride, carboplatin, and cisplatin. In addition many antibody-based therapeutic agents are administered to treat ovarian cancers, such as bevacizumab, olaparib and rucaparib camysylate.

Gemcitabine (i.e., 2′2′-difluoro 2′deoxycytidine, dFdC, dFdCyd, difluorodeoxycytidine hydrochloride or more specifically, gemcitabine hydrochloride) is a well known pyrimidine nucleoside. Gemcitabine is a hydrochloride salt of an analogue of the antimetabolite nucleoside deoxycytidine, which possesses anti-neoplastic activity. Gemcitabine is converted intracellularly to the active metabolites difluorodeoxycytidine di- and triphosphate (dFdCDP, dFdCTP). dFdCDP inhibits ribonucleotide reductase, thereby decreasing the deoxynucleotide pool available for DNA synthesis; dFdCTP is incorporated into DNA, resulting in DNA strand termination and apoptosis. Gemcitabine has the chemical structure 1-(2-oxo-4-amino-1,2-dihydropyrimidin-1-yl)-2-deoxy-2,2-difluororibose hydrochloride.

5-fluorouracil (i.e., 5-FU, or more specifically, 5-fluoro-1H-pyrimidine-2,4-dione) is a well known pyrimidine antagonist that is used in many adjuvant chemotherapeutic medicants, such as Carac® cream, Efudex®, Fluoroplex®, and Adrucil®. It is well established that 5-FU targets a critical enzyme, thymidylate synthase (TYMS or TS), which catalyzes the methylation of deoxyuridine monophosphate (dUMP) to deoxythymidine monophosphate (dTMP) an essential step in DNA biosynthesis. Danenberg P. V., Biochim. Biophys. Acta. (1977) 473(2):73-92.

Nevertheless, the existing cancer therapies are still in their infancy, with many hurdles still waiting to be improved or overcome. For example, it is well known that, although fairly efficacious in treating a variety of cancers, 5-FU and gemcitabine possess substantial toxicity and can elicit a host of adverse side effects. Moreover, tumor cells have been known to circumvent apoptotic pathways by developing resistance to common therapeutic agents, such as 5-FU and gemcitabine. See Gottesman M. M. et al., Nature Review's Cancer, (2002) 2(1):48-58. Thus, there would be a significant benefit in more efficacious, stable, and less toxic medications for the treatment of cancer.

### SUMMARY OF THE DISCLOSURE

Without being bound by any one particular theory, the present disclosure is premised on the discovery that replacing cytosine bases within the nucleotide sequences of microRNAs with gemcitabine increases microRNA efficacy as an anticancer therapeutic agent, when compared to certain known chemotherapeutic agents alone and/or the native microRNA molecule. The current disclosure demonstrates that nucleic acid compositions (i.e., a microRNA) of the present disclosure, which replace at least one cytosine base with a gemcitabine molecule, have exceptional efficacy as anti-cancer agents. Moreover, the data herein shows that contacting a cell with a modified microRNA composition of the present disclosure reduces tumorigenesis by, for example, reducing cancer cell growth and viability. Furthermore, it is shown that the modified microRNAs of the present disclosure retain target specificity, can be delivered without the use of harmful and ineffective delivery vehicles (e.g., nanoparticles), and exhibit enhanced potency and stability without abolishing the natural function of the native microRNA. Hence the present disclosure provides novel modified microRNA compositions with enhanced stability, potency, and target specificity for the treatment of cancer.

Therefore, in one aspect of the present disclosure nucleic acid compositions that include a modified microRNA nucleotide sequence having at least one cytosine base (C, C-bases) that has been replaced by a gemcitabine molecule are described. In certain embodiments, the modified microRNA has more than one, or exactly one cytosine that has been replaced by gemcitabine. In some embodiments, the modified microRNA nucleotide sequence replaces two, three, four, five or more cytosine bases with a gemcitabine molecule. In specific embodiments, all of the cytosine bases of a native microRNA have each been replaced by a gemcitabine molecule.

In a specific embodiment, the nucleic acid composition includes a modified native miR-194 nucleotide sequence that has been modified by replacing at least one of the cytosine bases with a gemcitabine molecule. More specifically, the nucleic acid composition contains at least the following native miR-194 nucleotide sequence: UGUAACAGCAACUCCAUGUGGA [SEQ ID NO. 1], wherein at least one, two, three, four or all of the cytosine bases are replaced by a gemcitabine molecule. In one instance, precisely one of the cytosine bases in the modified miR-194 nucleotide sequence is replaced by a gemcitabine molecule. In other instances, precisely or at least two cytosine bases in the modified miR-194 nucleotide sequence are each replaced by a gemcitabine molecule. In yet other instances, precisely or at least three cytosine bases in the modified miR-194 nucleotide sequence are each replaced by a gemcitabine molecule. In another instance, precisely or at least four cytosine bases in the modified miR-194 nucleotide sequence each replaced by a gemcitabine molecule. In specific embodiments, all of the cytosine bases in the modified miR-194 sequence are each replaced by a gemcitabine molecule. The modifications to miR-194 can be made in the guide strand or passenger strand of the native microRNA. In a preferred embodiment, the modifications to the miR-194 molecule are made to the guide strand.

In an exemplary embodiment, the nucleic acid composition of the present disclosure has a modified miR-194 nucleotide sequence of UGUAANAGNAANUNNAUGUGGA [SEQ ID NO. 2], wherein N is a gemcitabine molecule.

The present disclosure also shows that microRNAs having at least one uracil base (U, U-bases) replaced by a 5-halouracil, such as 5-fluorouracil (5-FU) and at least once cytosine base replaced by a gemcitabine molecule exhibits an improved therapeutic effect on cancer cells, when compared to the native microRNA alone or a microRNA modified by replacing at least one uracil base with 5-FU.

Therefore, in another aspect of the present disclosure nucleic acid compositions that include a modified microRNA nucleotide sequence having at least one uracil base replaced by a 5-halouracil and at least once cytosine base replaced by a gemcitabine molecule are describe. In certain embodiments, the modified microRNA has more than one, or exactly one uracil that has been replaced by a 5-halouracil and more than one, or exactly one cytosine that has been replaced by gemcitabine. In some embodiments, the modified microRNA nucleotide sequence replaces two, three, four or five uracil bases with a 5-halouracil and two, three, four or five cytosine bases with a gemcitabine molecule. In specific embodiments, all of the uracil bases of a native microRNA have been replaced by a 5-halouracil and all cytosine bases of the native microRNA have been replaced by a gemcitabine molecule.

In some embodiments, the 5-halouracil is, for example, 5-fluorouracil, 5-chlorouracil, 5-bromouracil, or 5-iodouracil. In specific embodiments, the 5-halouracil is 5-fluorouracil.

In certain embodiments, the modified microRNA nucleotide sequence includes more than one 5-halouracil whereby each of the 5-halouracils are the same. In other embodiments, the modified microRNA nucleotide sequence includes more than one 5-halouracil whereby each of the 5-halouracils is different. In other embodiments, the modified microRNA nucleotide sequence includes more than two 5-halouracils, whereby the modified microRNA nucleotide sequence includes a combination of different 5-halouracils.

In an exemplary embodiment of the present disclosure, a nucleic acid composition that contains a miR-194 nucleotide sequence that has been modified by replacing at least one of the uracil nucleotide bases with a 5-halouracil and replacing at least one of the cytosine nucleotide bases with a gemcitabine molecule is provided.

In one instance, precisely one of the cytosine bases in the native miR-194 nucleotide sequence is replaced by a gemcitabine molecule and precisely one of the uracil bases are replaced by a 5-halouracil. In other instances, precisely or at least two cytosine bases in the miR-194 nucleotide sequence are each replaced by a gemcitabine molecule and precisely or at least two of the uracil bases are each replaced by a 5-halouracil. In yet other instances, precisely or at least three cytosine bases in the miR-194 nucleotide sequence are each replaced by a gemcitabine molecule and precisely or at least three of the uracil bases are each replaced by a 5-halouracil. In another instance, precisely or at least four cytosine bases in the miR-194 nucleotide sequence each replaced by a gemcitabine molecule and precisely or at least four of the uracil bases are each replaced by a 5-halouracil. In specific embodiments, all of the cytosine bases in the miR-194 sequence are each replaced by a gemcitabine molecule and all of the uracil bases are each replaced by a 5-halouracil, such as 5-FU.

In an exemplary embodiment, the nucleic acid composition of the present disclosure has a modified miR-194 nucleotide sequence of U^{F}GU^{F}AANAGNAANU^{F}NNAU^{F}GU^{F}GGA [SEQ ID NO. 3], wherein N is a gemcitabine molecule and U^{F} is a halouracil, specifically 5-fluorouracil.

The present disclosure is also directed to formulations of a modified microRNA composition described herein or a formulation that includes combinations thereof, i.e., at least two different modified microRNAs. In certain embodiments, the formulations can include pharmaceutical preparations that comprise the above-described nucleic acid compositions and other known pharmacological agents, such as one or more pharmaceutically acceptable carriers.

The present disclosure reveals that the modified microRNAs each exhibit a potent efficacy as an anti-cancer therapeutic. Notably, each of the modified microRNA nucleic acid compositions tested reduce cancer cell viability, tumor growth and development.

Therefore, another aspect of the present disclosure is directed to a method for treating cancer that includes administering to a subject an effective amount of one or more of nucleic acid compositions described herein. In certain embodiments of the present methods, the nucleic acid compositions include a modified miR-194, wherein at least one, two, three, four, or more of the cytosine bases are replaced by a gemcitabine molecule.

In specific embodiments, the method includes administering a nucleic acid composition of the present disclosure to a subject having cancer or a predisposition to cancer, whereby the nucleic acid composition is a modified miR-194 molecule having the nucleic acid sequence UGUAANAGNAANUNNAUGUGGA [SEQ ID NO. 2], wherein N is a gemcitabine molecule.

In another embodiment, the present methods include administering a modified miR-194 having at least one, two, three, four, or more of the cytosine bases replaced by a gemcitabine molecule and at least one, two, three, four, or more of the uracil bases replaced by a halouracil, such as 5-fluorouracil. In a specific embodiment, the present method includes administration of a modified mir-194 that has each of the cytosine bases replaced by a gemicitabine molecule and each of the uracil nucleotide bases replaced by a 5-halouracil.

In specific embodiments, the present methods include administering a nucleic acid composition of the present disclosure to a subject having cancer or a predisposition to cancer, whereby the nucleic acid composition is a modified miR-194 molecule having the nucleic acid sequence U^{F}GU^{F}AANAGNAANU^{F}NNAU^{F}GU^{F}GGA [SEQ ID NO. 3], wherein N is a gemcitabine molecule and U^{F} is a halouracil, specifically 5-fluorouracil.

In some instances, the subject being treated by the present methods is a mammal. In certain embodiments, the subject being treated is a human, dog, horse, pig, mouse, or rat. In a specific embodiment, the subject is a human that has been diagnosed with cancer, or has been identified as having a predisposition to developing cancer. In some embodiments, the cancer being treated can be, for example, pancreatic, lung, ovarian cancer, breast or bladder cancer. In a specific embodiment, the cancer being treated is pancreatic cancer.

### BRIEF DESCRIPTION OF THE DRAWINGS

FIGS. 1A-1D. Chemical representation of exemplary modified microRNA nucleotide sequences of the present disclosure. (A) Chemical representation of the native miR-194 nucleotide sequence in which no C bases or U bases are replaced by gemcitabine or a halouracil, respectively (SEQ ID NO: 1). (B) Chemical representation of the native miR-194 nucleotide sequence in which all U bases are replaced by a halouracil (i.e., U^{F}), as set forth in SEQ ID NO: 4 (U^{F}GU^{F}AACAGCAACU^{F}CCAU^{F}GU^{F}GGA). (C) Chemical representation of miR-194 in which all cytosine bases are replaced with a gemcitabine molecule (X), as set forth in SEQ ID NO: 2. (D) Chemical representation of miR-194 nucleotide sequence in which all cytosine bases are replaced by a gemcitabine molecule and each uracil base is replaced by a 5-FU molecule, as set forth in SEQ ID NO: 3. The orientation of each exemplary modified microRNA depicted is provided by a 5′ to 3′ or 3′ to 5′ designation.

FIGS. 2A-D. Exemplary modified microRNA nucleic acids enter cancer cells and effectively reduce target protein expression. (A) Western blot showing miR-194 target SET8 and the ability of exemplary modified miR-194 compositions having all U bases replaced with 5-FU and all C bases replaced with gemcitabine (5-FU-Gem-miR-194, as set forth in SEQ ID NO: 3), and exemplary modified miR-194 composition having all C bases replaced with gemcitabine (Gem-mir-194, as set forth in SEQ ID NO: 2) to enter cells and inhibit target (SET8) expression in the presence of a transfections agent compared to that of control modified miR-194 (5-FU-miR-194 as set forth in SEQ ID NO: 4), and an unmodified miR-194 nucleic acid. (B) Western blot shows that cells transfected with the exemplary modified microRNAs of the present disclosure in the absence of a transfection agent enter the cell and inhibit SET8 expression, while control nucleic acid was unable to inhibit target expression. (C) Western blot showing another exemplary miR-194 target (BMI1) and the ability of exemplary modified miR-194 composition having all U bases replaced with 5-FU and all C bases replaced with gemcitabine (5-FU-Gem-miR-194, as set forth in SEQ ID NO: 3), and exemplary modified miR-194 composition having all C bases replaced with gemcitabine (Gem-mir-194, as set forth in SEQ ID NO: 2) to enter cells and inhibit target (BMI1) expression in the presence of a transfection agent compared to that of control modified miR-194 (5-FU-miR-194 as set forth in SEQ ID NO: 4), and an unmodified miR-194 nucleic acid. (D) Western blot shows that cells transfected with the exemplary modified microRNAs of the present disclosure in the absence of a transfection agent enter the cell and inhibit BMI1 expression, while unmodified miR-194 control nucleic acids were unable to inhibit target expression.

FIGS. 3A-3C. Graphs showing inhibition of pancreatic cancer cell viability in a dose dependent manner in 3 different pancreatic cancer cell lines (A) ASPC1, (B) PANC1 and (C) HS766T by exemplary modified miR-194 molecules. Exemplary modified miR-194 composition having all U bases replaced with 5-FU and all C bases replaced with gemcitabine (5-FU-Gem-miR-194, as set forth in SEQ ID NO: 3), and exemplary modified miR-194 composition having all C bases replaced with gemcitabine (Gem-mir-194, as set forth in SEQ ID NO: 2) inhibit pancreatic cancer cell viability when compared to exogenously expressed native miR-194 control, and modified miR-194 (5-FU-miR-194 as set forth in SEQ ID NO: 4).

FIG. 4. In vivo systemic treatment with exemplary modified microRNA nucleic acid compositions inhibits pancreatic cancer metastasis and tumor growth. A pancreatic cancer metastasis mouse model was established via tail vein injection of metastatic human pancreatic cancer cells. Four days after establishing metastasis, 80 μg of a modified miR-194 nucleic acid composition, as set forth in SEQ ID NOs: 2 were delivered by intravenous injection with a treatment frequency of one injection every other day for two weeks. The exemplary modified miR-194 nucleic acid was able to inhibit metastatic pancreatic cancer growth compared to control. Mice treated with modified miR-194 nucleic acids did not exhibit any toxicity.

TABLE 1. IC50 for each exemplary modified microRNAs in pancreatic cancer cell lines. In ASPC1 pancreatic cancer cells the IC50 for a modified miR-194 having all U bases replaced with 5-FU (5-FU-mIR-194, SEQ ID NO: 4) was 6.06 nM; the IC 50 for a modified miR-194 having all C bases replaced with gemcitabine (Gem-miR-194, as set forth in SEQ ID NO: 2) was 4.29 nM and the IC50 for a modified miR-194 having all U bases replaced with 5-FU and all C bases replaced with gemcitabine (5-FU-Gem-miR-194, as set forth in SEQ ID NO: 3) 5 was 2.88 nM. In PANC1 pancreatic cancer cells, the IC50 for a modified miR-194 having all U bases replaced with 5-FU (5-FU-mIR-194, SEQ ID NO: 4) was 16 nM; the IC50 for a modified miR-194 having all C bases replaced with gemcitabine (Gem-miR-194, as set forth in SEQ ID NO: 2) was 1.92 nM and the IC50 for a modified miR-194 having all U bases replaced with 5-FU and all C bases replaced with gemcitabine (5-FU-Gem-miR-194, as set forth in SEQ ID NO: 3) was 0.93 nM. In HS766T pancreatic cancer cells the IC50 for a modified miR-194 having all U bases replaced with 5-FU (5-FU-mIR-194, SEQ ID NO: 4) was 26.45 nM; the IC50 for a modified miR-194 having all C bases replaced with gemcitabine (Gem-miR-194, as set forth in SEQ ID NO: 2) was 3.57 nM and the IC50 for a modified miR-194 having all U bases replaced with 5-FU and all C bases replaced with gemcitabine (5-FU-Gem-miR-194, as set forth in SEQ ID NO: 3) 5 was 2.46 nM.

### DETAILED DESCRIPTION OF THE DISCLOSURE

The present disclosure provides nucleic acid compositions that incorporate one or more gemcitabine molecules. Without being bound by any one particular theory, surprisingly, the present disclosure reveals that the replacement of cytosine nucleotides within a microRNA oligonucleotide sequence with a gemcitabine molecule increases the ability of the microRNA to inhibit cancer development, progression and tumorigenesis. Moreover, the data herein shows that contacting cancer cells with a modified microRNA composition of the present disclosure reduces the viability of cancer cells in a dose dependent matter when compared to native microRNAs alone or microRNAs modified by replacing uracil bases with 5-FU. Furthermore, it is shown that the modified microRNAs of the present disclosure retain target specificity, can be delivered without the use of harmful and ineffective delivery vehicles (e.g., nanoparticles), and exhibit enhanced potency and stability without abolishing the natural function of the native microRNA. As such, the present disclosure provides various nucleic acid (e.g., microRNA) compositions having one or more gemcitabine molecules incorporated in their nucleic acid sequences and methods for using the same to treat cancer. The present disclosure further provides formulations, such as pharmaceutical compositions comprising the modified nucleic acid compositions, and methods for treating cancers that include administration of the same to a subject in need thereof.

### Nucleic Acid Compositions.

The term “microRNA” or “miRNA” or “miR” is used interchangeably to refer to small non-coding ribose nucleic acid (RNA) molecules that are capable of regulating the expression of genes through interacting with messenger RNA molecules (mRNA), DNA or proteins. Typically, microRNAs are composed of nucleic acid sequences of about 19-25 nucleotides (bases) and are found in mammalian cells. Mature microRNA molecules are single stranded RNA molecules processed from double stranded precursor transcripts that form local hairpin structures. The hairpin structures are typically cleaved by the Dicer enzyme to form a double stranded microRNA duplex. See, e.g., Bartel, Cell, (2004) 116 pp. 281-297. The term microRNA as used herein incorporates both the duplex (i.e., double stranded miRs) and single stranded miRs (i.e., mature miRs) in both the 5′ to 3′ direction and complementary strand in the 3′ to 5′ direction. In specific embodiments, modified miRs of the present disclosure are composed of single stranded mature MiRs.

Usually, one of the two strands of a microRNA duplex is packaged in a microRNA ribonucleoprotein complex (microRNP). A microRNP in, for example, humans, also includes the proteins eIF2C2/Argonaute (Ago2), the helicase Gemin3, and Gemin 4. Other members of the Argonaute protein family, such as Ago1, 3, and 4, also associate with microRNAs and form microRNPs.

The term “modified microRNA”, “modified miRNA”, “modified miR” or “mimic” are used interchangeably herein to refer to a microRNA that differs from the native or endogenous microRNA (unmodified microRNA) polynucleotide. More specifically, in the present disclosure a modified microRNA differs from the unaltered or unmodified microRNA nucleic acid sequence by one or more base. In some embodiments of the present disclosure, a modified microRNA of the present disclosure includes at least one cytosine (C) nucleotide base replaced by a gemcitabine molecule. In other embodiments, a modified microRNA of the present disclosure includes at least one uracil (U) nucleotide base replaced by a 5-halouracil and at least one cytosine (C) nucleotide base replaced by a gemcitabine molecule.

The term “gemcitabine” as used herein is synonymous with 2′-Deoxy-2′,2′-difluorocytidine, 2′,2′-Difluorodeoxycytidine, 4-amino-1-((2R,4R,5R)-3,3-difluoro-4-hydroxy-5-(hydroxymethyl)-tetrahydrofuran-2-yl)pyrimidin-2(1H)-one, gemcitabine hydrochloride, dFdC, dFdCyd, and difluorodeoxycytidine hydrochloride. Gemcitabine is a nucleoside (pyrimidine) analog used as chemotherapy. Gemcitabine is marketed as Gemzar®. Gemcitabine has the following structure:

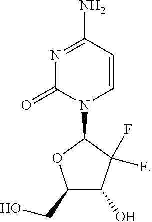 [SMILES: Nc1ccn([C@@H]2O[C@H](CO)[C@@H](O)C2(F)F)c(=O)n1]

Gemcitabine is known to arrest tumor growth by incorporating within the DNA during replication. Gemcitabine is approved to treat various types of cancer including, non-small cell lung cancer, pancreatic cancer, bladder cancer, breast cancer, and ovarian cancer.

In one aspect of the present disclosure, nucleic acid compositions that include a modified microRNA nucleotide sequence having at least one cytosine base (C) that has been replaced with a gemcitabine molecule are described. As further discussed herein, the nucleic acid compositions of the present disclosure are useful, at least, in the treatment of cancer. In particular, the exemplary modified microRNAs of the present disclosure have been shown herein to be effective in the treatment of pancreatic cancer.

In certain embodiments, the modified microRNA has more than one, or exactly one cytosine that has been replaced by gemcitabine. In some embodiments, the modified microRNA nucleotide sequence replaces two, three, four or five cytosine bases with a gemcitabine molecule. In specific embodiments, all of the cytosine bases of a native microRNA have each been replaced by a gemcitabine molecule.

In a specific embodiment, the nucleic acid composition includes a modified native miR-194 nucleotide sequence that has been modified by replacing at least one of the cytosine bases with a gemcitabine molecule.

The term “miR-194”, as used herein, is meant to be synonymous with the terms “microRNA-194” or “miRNA-194” and refers to an oligonucleotide having the following nucleotide sequence: UGUAACAGCAACUCCAUGUGGA [SEQ ID NO. 1]. The foregoing nucleotide sequence is herein referred to as a miR-194 unmodified (i.e., “native”) sequence unless otherwise specified. In some embodiments, miR-194 may be referred to in the field as hsa-miR-194 with accession number MI0000488 or M10000732 for the stem loop containing double stranded microRNA; hsa-miR-194-5p for the mature miR 5′ to 3′ strand as set forth in accession number MIMAT0000460; and hsa-miR-194-3p for the 3′ to 5′ complementary strand of a duplex molecule as set forth by accession number MIMAT0004671. MiR-194 is well known and has been studied in detail. See, e.g., Lagos-Quintana M, et al., RNA. 9: pp. 175-179 (2003). As is the case for the above, modified microRNAs, methods for creating a miR-194 mimics are known by those of ordinary skill in the art. Unless otherwise stated, all such modified miR-194 nucleic acid forms are herein considered to be within the scope of the term “miR-194 mimic”, as used herein.

Generally, a modified miR-194 (i.e., miR-194 mimic) contains no more than one, two, three, four, or five additional nucleotides covalently appended to the miR-194 native sequence, wherein the additional bases are independently selected from C, U, G, and A, or the additional bases may be exclusively one of C, U, G or A. Typically, the miR-194 mimic is used in single-strand form, but double-stranded versions are also considered herein.

More specifically, the modified microRNA composition contains at least the native miR-194 nucleotide sequence, wherein at least one, two, three, four or all of the cytosine bases are replaced by a gemcitabine molecule. In one instance, precisely one of the cytosine bases in the native miR-194 nucleotide sequence is replaced by a gemcitabine molecule. In other instances, precisely or at least two cytosine bases in the native miR-194 nucleotide sequence are each replaced by a gemcitabine molecule. In yet other instances, precisely or at least three cytosine bases in the native miR-194 nucleotide sequence are each replaced by a gemcitabine molecule. In another instance, precisely or at least four cytosine bases in the miR-194 nucleotide sequence are each replaced by a gemcitabine molecule. In specific embodiments, all of the cytosine bases in the guide strand of the native miR-194 sequence are each replaced by a gemcitabine molecule.

In an exemplary embodiment, the nucleic acid composition of the present disclosure has a modified miR-194 nucleotide sequence of UGUAANAGNAANUNNAUGUGGA [SEQ ID NO. 2], wherein N is a gemcitabine molecule.

The present disclosure also shows that microRNAs having at least one uracil base replaced by a 5-halouracil, such as 5-fluorouracil (5-FU) and at least once cytosine base replaced by a gemcitabine molecule exhibits an improved therapeutic effect on cancer cells, when compared to the native microRNA alone or a microRNA modified by replacing at least one uracil base with 5-FU.

Therefore, in the present disclosure also provides nucleic acid compositions that include a modified microRNA having at least one uracil base replaced by a 5-halouracil and at least once cytosine base replaced by a gemcitabine molecule. In certain embodiments, the modified microRNA has more than one, or exactly one uracil that has been replaced by a 5-halouracil and more than one, or exactly one cytosine that has been replaced by gemcitabine. In some embodiments, the modified microRNA nucleotide sequence replaces two, three, four or five uracil bases with a 5-halouracil and two, three, four or five cytosine bases with a gemcitabine molecule. In specific embodiments, all of the uracil bases of a native microRNA have been replaced by a 5-halouracil and all cytosine bases of the native microRNA have been replaced by a gemcitabine molecule.

In some embodiments, the 5-halouracil is, for example, 5-fluorouracil, 5-chlorouracil, 5-bromouracil, or 5-iodouracil. In specific embodiments, the 5-halouracil is 5-fluorouracil.

In certain embodiments, the modified microRNA nucleotide sequence includes more than one 5-halouracil whereby each of the 5-halouracils are the same. In other embodiments, the modified microRNA nucleotide sequence includes more than one 5-halouracil whereby each of the 5-halouracils is different. In other embodiments, the modified microRNA nucleotide sequence includes more than two 5-halouracils, whereby the modified microRNA nucleotide sequence includes a combination of different 5-halouracils.

In some embodiments, the nucleic acid compositions contain a nucleotide sequence that has been modified by derivatizing at least one of the uracil nucleobases at the 5-position with a group that provides a similar effect as a halogen atom. In some embodiments, the group providing the similar effect has a similar size in weight or spatial dimension to a halogen atom, e.g., a molecular weight of up to or less than 20, 30, 40, 50, 60, 70, 80, 90, or 80 g/mol. In certain embodiments, the group providing a similar effect as a halogen atom may be, for example, a methyl group, trihalomethyl (e.g., trifluoromethyl) group, pseudohalide (e.g., trifluoromethanesulfonate, cyano, or cyanate) or deuterium (D) atom. The group providing a similar effect as a halogen atom may be present in the absence of or in addition to a 5-halouracil base in the microRNA nucleotide sequence.

In an exemplary embodiment of the present disclosure, a nucleic acid composition that contains a miR-194 nucleotide sequence that has been modified by replacing at least one of the uracil nucleotide bases with a 5-halouracil and replacing at least one of the cytosine nucleotide bases with a gemcitabine molecule is provided. In one instance, precisely one of the cytosine bases of the native miR-194 nucleotide sequence is replaced by a gemcitabine molecule and precisely one of the uracil bases are replaced by a 5-halouracil. In other instances, precisely or at least two cytosine bases in the native miR-194 nucleotide sequence are each replaced by a gemcitabine molecule and precisely or at least two of the uracil bases are each replaced by a 5-halouracil. In yet other instances, precisely or at least three cytosine bases in the native miR-194 nucleotide sequence are each replaced by a gemcitabine molecule and precisely or at least three of the uracil bases are each replaced by a 5-halouracil. In another instance, precisely or at least four cytosine bases in the native miR-194 nucleotide sequence are each replaced by a gemcitabine molecule and precisely or at least four of the uracil bases are each replaced by a 5-halouracil. In specific embodiments, all of the cytosine bases in guide strand of the native miR-194 sequence are each replaced by a gemcitabine molecule and all of the uracil bases are each replaced by a 5-halouracil, such as 5-FU.

In an exemplary embodiment, the nucleic acid composition of the present disclosure has a modified miR-194 nucleotide sequence of U^{F}GU^{F}AANAGNAANU^{F}NNAU^{F}GU^{F}GGA [SEQ ID NO. 3], wherein N is a gemcitabine molecule and U^{F} is a halouracil, specifically 5-fluorouracil.

The modified microRNA nucleic acid compositions described herein can be synthesized using any of the well known methods for synthesizing nucleic acids. In particular embodiments, the nucleic acid compositions are produced by automated oligonucleotide synthesis, such as any of the well-known processes using phosphoramidite chemistry. To introduce one or more gemcitabine molecules or a 5-halouracil base in a modified miR sequence (e.g., miR-194 sequence), a gemcitabine or 5-halouracil nucleoside phosphoramidite can be included as a precursor base, along with the phosphoramidite derivatives of nucleosides containing natural bases (e.g., A, U, G, and C) to be included in the nucleic acid sequence.

In some embodiments, the nucleic acid compositions of the present disclosure may be produced biosynthetically, such as by using in vitro RNA transcription from plasmid, PCR fragment, or synthetic DNA templates, or by using recombinant (in vivo) RNA expression methods. See, e.g., C. M. Dunham et al., Nature Methods, (2007) 4(7), pp. 547-548. The modified microRNA sequences of the present disclosure (e.g., miR-194 sequence) may be further chemically modified such as by functionalizing with polyethylene glycol (PEG) or a hydrocarbon or a targeting agent, particularly a cancer cell targeting agent, such as folate, by techniques well known in the art. To include such groups, a reactive group (e.g., amino, aldehyde, thiol, or carboxylate group) that can be used to append a desired functional group may first be included in the oligonucleotide sequence. Although such reactive or functional groups may be incorporated onto the as-produced nucleic acid sequence, reactive or functional groups can be more facilely included by using an automated oligonucleotide synthesis in which non-nucleoside phosphoramidites containing reactive groups or reactive precursor groups are included.

### Modified Nucleic Acid Formulations

The present disclosure reveals that the modified microRNAs each exhibit a potent efficacy as an anti-cancer therapeutic. Notably, each of the modified microRNA nucleic acid compositions tested reduce cancer cell viability, tumor growth and development in a dose dependent manner.

As such, the present disclosure is also directed to formulations of the modified microRNA nucleic acid compositions described herein. For example, the present nucleic acid compositions can be formulated for pharmaceutical uses. In certain embodiments, a formulation is a pharmaceutical composition containing a nucleic acid composition described herein and a pharmaceutically acceptable carrier.

In some embodiments, a formulation of the present disclosure comprises a modified miR-194 nucleic acid having at least one cytosine base replaced by a gemcitabine molecule, a modified miR-194 nucleic acid having at least one cytosine base replaced by a gemcitabine molecule and at least one uracil base replaced by a halouracil, or a combination thereof and a pharmaceutically acceptable carrier.

More specifically, one or more of the modified microRNA nucleic acids set forth in the following nucleotide sequences can be formulated for pharmaceutical application and use; UGUAAXAGXAAXUXXAUGUGGA [SEQ ID NO. 2] or U^{F}GU^{F}AAXAGXAAXU^{F}XXAU^{F}GU^{F}GGA [SEQ ID NO. 3].

The term “pharmaceutically acceptable carrier” is used herein as synonymous with a pharmaceutically acceptable diluent, vehicle, or excipient. Depending on the type of pharmaceutical composition and intended mode of administration, the nucleic acid composition may be dissolved or suspended (e.g., as an emulsion) in the pharmaceutically acceptable carrier. The pharmaceutically acceptable carrier can be any of those liquid or solid compounds, materials, compositions, and/or dosage forms which are, within the scope of sound medical judgment, suitable for use in contact with tissues of a subject. The carrier should be “acceptable” in the sense of being not injurious to the subject it is being provided to and is compatible with the other ingredients of the formulation, i.e., does not alter their biological or chemical function.

Some, non-limiting examples, of materials which can serve as pharmaceutically acceptable carriers include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. The pharmaceutically acceptable carrier may also include a manufacturing aid (e.g., lubricant, talc magnesium, calcium or zinc stearate, or stearic acid), a solvent, or encapsulating material. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19^{th} Ed. Mack Publishing Company, Easton, Pa., (1995).

In some embodiments, the pharmaceutically acceptable carrier may include diluents that increase the bulk of a solid pharmaceutical composition and make the pharmaceutical dosage form easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

The nucleic acid compositions of the present disclosure may be formulated into compositions and dosage forms according to methods known in the art. In certain embodiments, the formulated compositions may be specially formulated for administration in solid or liquid form, including those adapted for the following: (1) oral administration, for example, tablets, capsules, powders, granules, pastes for application to the tongue, aqueous or non-aqueous solutions or suspensions, drenches, or syrups; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

In some embodiments, the formulations of the present disclosure include a solid pharmaceutical agent that is compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in a subject's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Therefore, in certain embodiments, glidants can be added to formulations to improve the flowability of a non-compacted solid agent and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

A formulated pharmaceutical composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried and then screened and/or milled to the desired particle size. The granulate may then be tableted, or other excipients may be added prior to tableting, such as a glidant and/or a lubricant. A tableting composition may be prepared conventionally by dry blending. For example, the blended composition of the actives and excipients may be compacted into a slug or a sheet and then comminuted into compacted granules. The compacted granules may subsequently be compressed into a tablet.

In other embodiments, as an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting. A capsule filling may include any of the aforementioned blends and granulates that were described with reference to tableting; however, they are not subjected to a final tableting step.

In liquid pharmaceutical compositions of the present disclosure, the agent and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. The liquid formulation may be used as an injectable, enteric, or emollient type of formulation. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

In some embodiments, liquid pharmaceutical compositions of the present disclosure may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum. In other embodiments, the liquid composition of the present disclosure may also contain a buffer, such as gluconic acid, lactic acid, citric acid or acetic acid, sodium gluconate, sodium lactate, sodium citrate, or sodium acetate.

Sweetening agents, such as sorbitol, saccharin, sodium saccharin, sucrose, aspartame, fructose, mannitol and invert sugar, may be added to certain formulations of the present disclosure to improve the taste. Flavoring agents and flavor enhancers may make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol and tartaric acid.

Preservatives and chelating agents, such as alcohol, sodium benzoate, butylated hydroxy toluene, butylated hydroxyanisole and ethylenediamine tetraacetic acid, may be added at levels safe for ingestion to improve storage stability. Solid and liquid compositions may also be dyed using any pharmaceutically acceptable colorant to improve their appearance and/or facilitate patient identification of the product and unit dosage level.

A dosage formulation of the present disclosure may be a capsule containing the composition, for example, a powdered or granulated solid composition of the disclosure, within either a hard or soft shell. The shell may be made from gelatin and optionally contain a plasticizer such as glycerin and sorbitol, and an opacifying agent or colorant.

### Methods for Treating Cancer

As stated above, the modified microRNA nucleic acid compositions of the present disclosure and formulations thereof show unexpected and exceptional anti-cancer activity when compared to that exhibited by exogenous expression of a corresponding unmodified native microRNA and/or other known cancer therapies. Therefore, another aspect of the present disclosure provides a method for treating cancer in a mammal by administering to the mammal an effective amount of one or more of the modified microRNA nucleic acid compositions of the present disclosure, or formulations thereof.

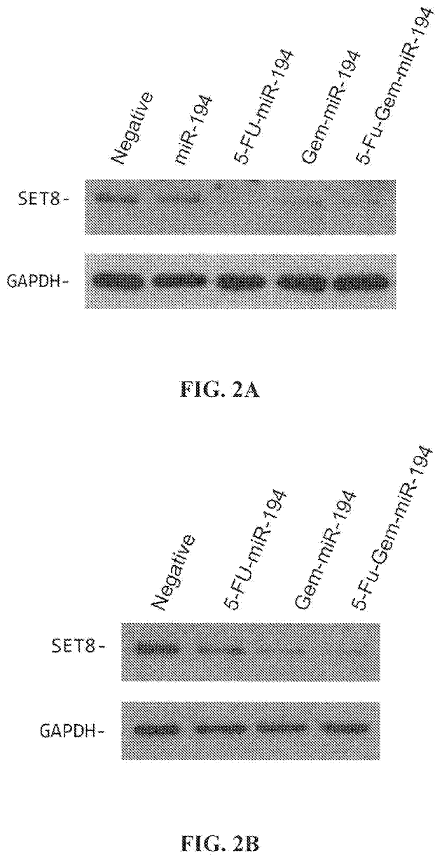

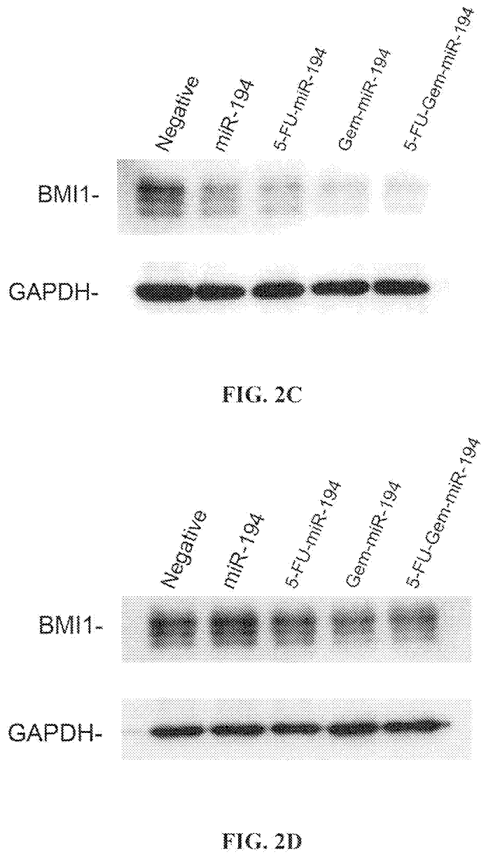

As shown in FIGS. 2A through 2D, exemplary modified microRNA nucleic acids of the present disclosure, i.e., modified miR-194, suppress SET8 protein expression (FIGS. 2A and 2B, BMI1 protein expression (FIGS. 2C and 2D) and activity in the cancer cells. More specifically, FIGS. 2A-2D show that modified microRNAs having all C bases replaced with gemcitabine (Gem-miR-194, as set forth in SEQ ID NO: 2) enter cancer cells with or without a transfection agent and inhibit SET8 and BMI1. Furthermore, modified microRNAs having all C bases replaced with gemcitabine and all U bases replaced with 5-FU (5-FU-Gem-miR-194, as set forth in SEQ ID NO: 3) are capable entering cancer cells with or without a transfection agent to inhibit SET8 or BMI1.

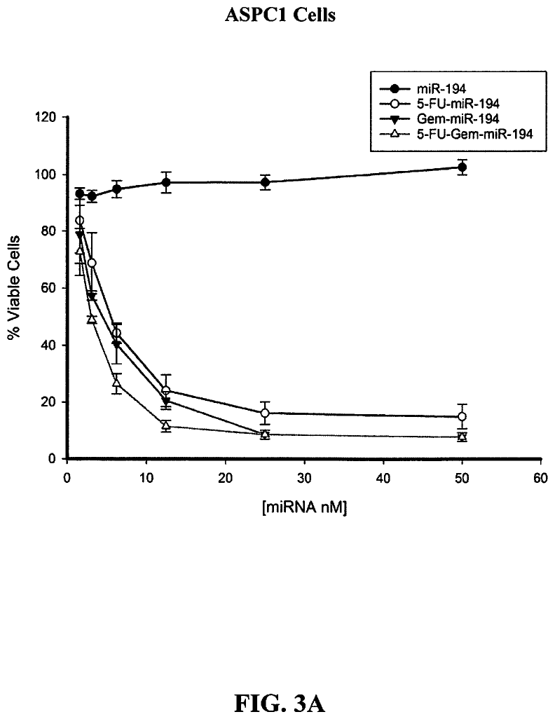

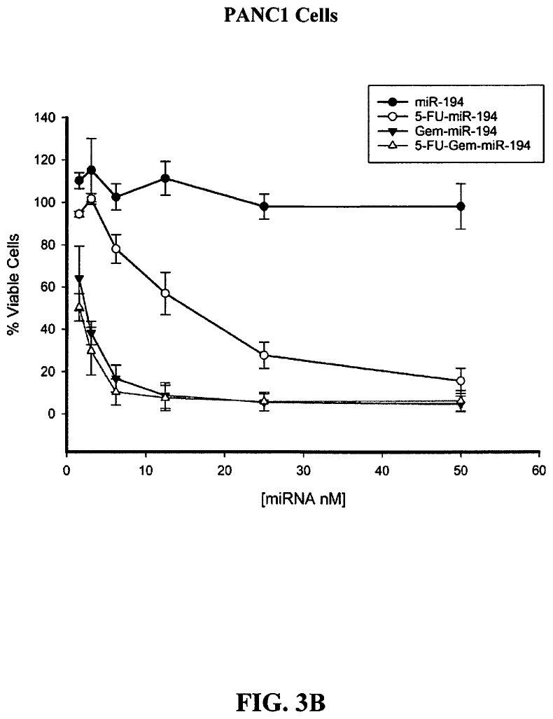

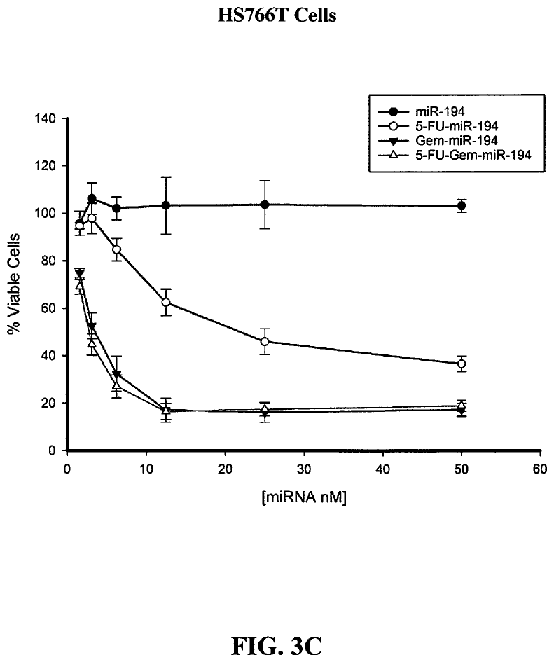

In addition and as shown in FIGS. 3A-3C, all of the exemplary modified microRNA's described herein reduce pancreatic cancer cell viability. More specifically, modified microRNAs having all C bases replaced with gemcitabine (Gem-miR-194, as set forth in SEQ ID NO: 2) reduce pancreatic cancer cell viability in 3 different pancreatic cancer cell lines (i.e., PANC1, ASPC1 and HS766T) in a dose dependent manner. Similarly, modified microRNAs having all C bases replaced with gemcitabine and all U bases replaced with 5-FU (5-FU-Gem-miR-194, as set forth in SEQ ID NO: 3) inhibit pancreatic cancer cell viability in all pancreatic cancer models tested in a dose dependent manner.

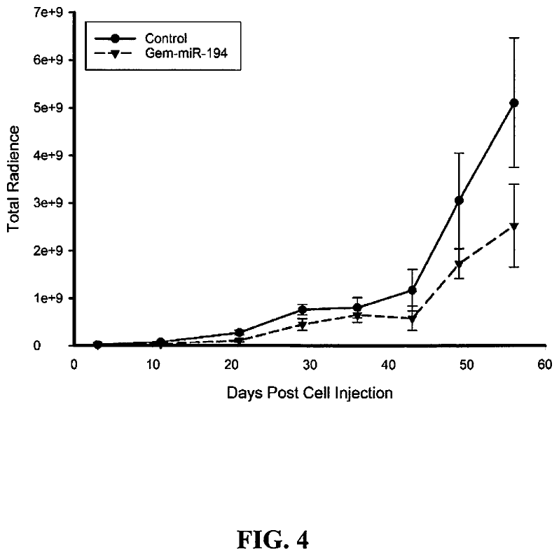

Moreover, the present modified miR compositions were tested and found to be therapeutically effective in vivo. For example, FIG. 4 show that intravenous treatment with two exemplary modified microRNA's of the present disclosure (e.g., modified miR-194 as set forth in SEQ ID NO: 2) effectively treat cancer (e.g., pancreatic cancer) by inhibiting tumor growth in vivo.

Therefore, the disclosed methods for treating cancer include administering one or more modified nucleic acid compositions of the present disclosure (e.g., a modified microRNA, such as a modified miR-194 nucleic acid to a subject. In certain embodiments, the nucleic acid composition can be administered as a formulation that includes a nucleic acid composition and one or more pharmaceutical carriers.

In specific embodiments, the nucleic acid compositions of the present disclosure can be administered in the absence of a delivery vehicle or pharmaceutical carrier (i.e., naked). See, for example, FIGS. 2B and 2D.

The term “subject” as used herein refers to any mammal. The mammal can be any mammal, although the methods herein are more typically directed to humans. The phrase “subject in need thereof” as used herein is included within the term subject and refers to any mammalian subject in need of a treatment, particularly cancer or has a medically determined elevated risk of a cancerous or pre-cancerous condition. In specific embodiments, the subject includes a human cancer patient.

The terms “treatment” “treat” and “treating” are synonymous with the term “to administer an effective amount”. These terms shall mean the medical management of a subject with the intent to cure, ameliorate, stabilize, reduce one or more symptoms of or prevent a disease, pathological condition, or disorder such as cancer. These terms, are used interchangeably and include the active treatment, that is, treatment directed specifically toward the improvement of a disease, pathological condition, or disorder, and also include causal treatment, that is, treatment directed toward removal of the cause of the associated disease, pathological condition, or disorder. In addition, treating includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount. In a specific embodiment, treatment of a disease, such as a cancer includes inhibiting proliferation of cancer cells. In some embodiments, the treatment of a cancer can be determined by detecting a reduction in the amount of proliferating cancer cells in a subject, a reduction in tumor growth or tumor size.

In certain embodiments, the nucleic acid compositions of the present disclosure are used to treat cancer.

The term “cancer”, as used herein, includes any disease caused by uncontrolled division and growth of abnormal cells, including, for example, the malignant and metastatic growth of tumors. The term “cancer” also includes pre-cancerous conditions or conditions characterized by an elevated risk of a cancerous or pre-cancerous condition. The cancer or pre-cancer (neoplastic condition) can be located in any part of the body, including the internal organs and skin. As is well known, cancer spreads through a subject by invading the normal, non-cancerous tissue surrounding the tumor, via the lymph nodes and vessels, and by blood after the tumor invades the veins, capillaries and arteries of a subject. When cancer cells break away from the primary tumor (“metastasize”), secondary tumors arise throughout an afflicted subject forming metastatic lesions.

Some non-limiting examples of applicable cancer cells for treatment using the present methods include the lungs, breast, pancreas, bladder and ovaries. The cancer or neoplasm can also include the presence of one or more carcinomas, sarcomas, lymphomas, blastomas, or teratomas (germ cell tumors).

In some embodiments, the subject has pancreatic cancer, or has a medically determined elevated risk of getting pancreatic cancer such as, for example, being diagnosed with chronic pancreatitis.

In certain embodiments, a subject of the present disclosure has breast cancer, or has a medically determined elevated risk of getting breast cancer. In specific embodiments, the breast cancer is triple negative breast cancer, ductal carcinoma or lobal carcinoma.

In other embodiments, the subject has ovarian cancer, or has a medically determined elevated risk of getting ovarian cancer.

In yet other embodiments, the subject has bladder cancer, or has a medically determined elevated risk of getting bladder cancer.

In a specific example, the modified microRNAs of the present disclosure are used to treat pancreatic cancer. As shown in FIGS. 3A-C and 4, each of modified miR-194 microRNAs can be used to treat pancreatic cancer. Pancreatic cancer arises from precursor lesions called pancreatic intraepithelial neoplasia, or PanINs. These lesions are typically located in the small ducts of the exocrine pancreas, and depending on the extent of cytologic atypia may be classified as low-grade dysplasia, moderate dysplasia or high-grade dysplasia lesions. Such lesions typically show that activating mutations in the KRAS gene present, along with certain inactivating mutations in CDKN2A, IP53 and SAMD4. Collectively, these genetic mutations lead to the formation of an infiltrating cancer. Pancreatic cancer is staged based on size of the primary tumor and whether it has grown outside of the pancreas into surrounding organs; whether the tumor has spread to the nearby lymph nodes, and whether it has metastasized to other organs of the body (e.g., liver, lungs, abdomen). This information is then combined and used to provide the specific stage, i.e., 0, 1A, 1B, 2A, 2B, 3 and 4. For stage zero (0), the pancreatic tumor is confined to the top layers of pancreatic duct cells and has not invaded deeper tissues. The primary tumor has not spread outside of the pancreas such as in pancreatic carcinoma in situ or pancreatic intraepithelial neoplasia III. A stage 1A pancreatic tumor is typically confined to the pancreas and is 2 cm across or smaller. Further a stage 1A pancreatic tumor has not spread to nearby lymph nodes or distant sites. A stage 1B pancreatic tumor confined to the pancreas and is larger than 2 cm across. A stage 1B pancreatic tumor has not spread to nearby lymph nodes or distant sites. Stage 2A pancreatic tumors exhibit a tumor growing outside the pancreas but not into major blood vessels or nerves, but the cancer has not spread to nearby lymph nodes or distant sites. A subject exhibiting stage 2B pancreatic cancer presents a tumor is either confined to the pancreas or growing outside the pancreas but not into major blood vessels or nerves, but has spread to nearby lymph nodes. A subject exhibiting stage 3 pancreatic cancer presents a tumor that is growing outside the pancreas into major blood vessels or nerves, but has spread to distant sites. Stage 4 pancreatic cancer has metastasized to distant cites, lymph nodes and organs.

According to the present disclosure, methods of treating cancer include administration of one or more nucleic acid compositions of the present by any of the routes commonly known in the art. This includes, for example, (1) oral administration; (2) parenteral administration, for example, by subcutaneous, intramuscular or intravenous injection; (3) topical administration; or (4) intravaginal or intrarectal administration; (5) sublingual or buccal administration; (6) ocular administration; (7) transdermal administration; (8) nasal administration; and (9) administration directly to the organ or cells in need thereof.

In specific embodiments, the modified microRNA compositions of the present disclosure are administered to a subject by injection. In one embodiment, a therapeutically effective amount of a modified microRNA composition is injected intravenously. In another embodiment, a therapeutically effective amount of a modified microRNA composition is injected intraperitoneally.

The amount (dosage) of nucleic acid compositions of the present disclosure being administered depends on several factors, including the type and stage of the cancer, presence or absence of an auxiliary or adjuvant drug, and the subject's weight, age, health, and tolerance for the agent. Depending on these various factors, the dosage may be, for example, about 2 mg/kg of body weight, about 5 mg/kg of body weight, about 10 mg/kg of body weight, about 15 mg/kg of body weight, about 20 mg/kg of body weight, about 25 mg/kg of body weight, about 30 mg/kg of body weight, about 40 mg/kg of body weight, about 50 mg/kg of body weight, about 60 mg/kg of body weight, about 70 mg/kg of body weight, about 80 mg/kg of body weight, about 90 mg/kg of body weight, about 100 mg/kg of body weight, about 125 mg/kg of body weight, about 150 mg/kg of body weight, about 175 mg/kg of body weight, about 200 mg/kg of body weight, about 250 mg/kg of body weight, about 300 mg/kg of body weight, about 350 mg/kg of body weight, about 400 mg/kg of body weight, about 500 mg/kg of body weight, about 600 mg/kg of body weight, about 700 mg/kg of body weight, about 800 mg/kg of body weight, about 900 mg/kg of body weight, or about 1000 mg/kg of body weight, wherein the term “about” is generally understood to be within f 10%, 5%, 2%, or 1% of the indicated value. The dosage may also be within a range bounded by any two of the foregoing values. Routine experimentation may be used to determine the appropriate dosage regimen for each patient by monitoring the compound's effect on the cancerous or pre-cancerous condition, or effect on microRNA expression level or activity (e.g., miR-194, or effect on a target thereof, such as SET8 and/or BMI1 level or activity, or the disease pathology, all of which can be frequently and easily monitored according to methods known in the art. Depending on the various factors discussed above, any of the above exemplary doses of nucleic acid can be administered once, twice, or multiple times per day.

The ability of the nucleic acid compositions described herein, and optionally, any additional chemotherapeutic agent for use with the current methods can be determined using pharmacological models well known in the art, such as cytotoxic assays, apoptosis staining assays, xenograft assays, and binding assays.

The nucleic acid compositions described herein may or may not also be co-administered with one or more chemotherapeutic agents, which may be auxiliary or adjuvant drugs different from a nucleic composition described herein.

As used herein, “chemotherapy” or the phrase a “chemotherapeutic agent” is an agent useful in the treatment of cancer. Chemotherapeutic agents useful in conjunction with the methods described herein include, for example, any agent that modulates BMI1, either directly or indirectly. Examples of chemotherapeutic agents include: anti-metabolites such as methotrexate and fluoropyrimidine-based pyrimidine antagonist, 5-fluorouracil (5-FU) (Carac® cream, Efudex®, Fluoroplex®, Adrucil®) and S-1; antifolates, including polyglutamatable antifolate compounds; raltitrexed (Tomudex®), GW1843 and pemetrexed (Alimta®) and non-polyglutamatable antifolate compounds; nolatrexed (Thymitaq®), plevitrexed, BGC945; folic acid analogs such as denopterin, methotrexate, pteropterin, trimetrexate; and purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine. In a specific embodiment of the current disclosure, the chemotherapeutic agent is a compound capable of inhibiting the expression or activity of genes, or gene products involved in signaling pathways implicated in aberrant cell proliferation or apoptosis, such as, for example, YAP1, BMI1, SET8, DCLK1, BCL2, thymidylate synthase or E2F3; and pharmaceutically acceptable salts, acids or derivatives of any of the above.

In other embodiments, the chemotherapy can be any of the following cancer drugs, such as one or more of methotrexate, doxorubicin, cyclophosphamide, cis-platin, oxaliplatin, bleomycine, vinblastine, gemcitabine, vincristine, epirubicin, folinic acid, paclitaxel, and docetaxel. The chemotherapeutic agent may be administered before, during, or after commencing therapy with the nucleic acid composition.

In some embodiments, the chemotherapeutic agent is an anti-cancer drug, or a tissue sensitizer or other promoter for an anti-cancer drug. In some embodiments, the co-drug may be another nucleic acid, or another miRNA, such as a microRNA mimic of the present disclosure, gemcitabine or free 5-FU.

In a specific embodiment, the other nucleic acid is a short hairpin RNA (shRNA), siRNA, or nucleic acid complementary to a portion of the BCL2 3′UTR.

In some embodiments, the chemotherapeutic agent is a co-drug.

Set domain-containing protein 8, SET8 or SETD8 (GenBank AF287261) is a lysine methyltransferase that predominately monomethylates lysine-20 of histone H5. SET8 modulates transcriptional regulation, heterochromatin formation, genomic stability, cell cycle progression and development. See Yang, F., et al. EMBO J. (2012) 31: pp. 110-123. Therefore, any drug that inhibits the expression of SET8 may be considered herein as a co-drug.

Polycomb complex protein BMI-1, BMI1 (RefSeq, NM_005180.8, NP_005171.4 encodes a ring finger protein that is major component of the polycomb group complex 1 (PRC1). This complex functions through chromatin remodeling as an essential epigenetic repressor of multiple regulatory genes involved in embryonic development and self-renewal in somatic stem cells. The BMI1 protein plays a central role in DNA damage repair. The BMI1 gene is an oncogene and aberrant expression is associated with numerous cancers and is associated with resistance to certain chemotherapies. Therefore, any drug that inhibits the expression of SET8 may be considered herein as a co-drug.

E2F transcription factor 3, E2F3 (RefSeq NG_029591.1, NM_001243076.2, NP_001230005.1) is a transcription factor that binds DNA and interacts with effector proteins, including but not limited to, retinoblastoma protein to regulate the expression of genes involved in cell cycle regulation. Therefore, any drug that inhibits the expression of E2F3 may be considered herein as a co-drug.

B-cell lymphoma 2 (BCL2), (RefSeq NG_009361.1, NM_000633, NP_000624) including isoform a (NM_000633.2, NP_000624.2) and β NM_000657.2, NP_000648.2 thereof, are encoded by the Bcl-2 gene, which is a member of the BCL2 family of regulator proteins that regulate mitochondria regulated cell death via the intrinsic apoptosis pathway. BCL2 is an integral outer mitochondrial membrane protein that blocks the apoptotic death of cell cells by binding BAD and BAK proteins. Non-limiting examples of BCL2 inhibitors include antisense oligonucleotides, such as Oblimersen (Genasense; Genta Inc.), BH3 mimetic small molecule inhibitors including, ABT-737 (Abbott Laboratories, Inc.), ABT-199 (Abbott Laboratories, Inc.), and Obatoclax (Cephalon Inc.). Any drug that inhibits the expression of BCL2 may be considered herein as a co-drug.

Thymidylate synthase (RefSeq: NG_028255.1, NM_001071.2, NP_001062.1) is a ubiquitous enzyme, which catalyses the essential methylation of dUMP to generate dTMP, one of the four bases which make up DNA. The reaction requires CH H₄-folate as a cofactor, both as a methyl group donor, and uniquely, as a reductant. The constant requirement for CH H₄-folate means that thymidylate synthase activity is strongly linked to the activity of the two enzymes responsible for replenishing the cellular folate pool: dihydrofolate reductase and serine transhydroxymethylase. Thymidylate synthase is a homodimer of 30-35 kDa subunits. The active site binds both the folate cofactor and the dUMP substrate simultaneously, with the dUMP covalently bonded to the enzyme via a nucleophilic cysteine residue (See, Carreras et al, Annu. Rev. Biochem., (1995) 64:721-762). The thymidylate synthase reaction is a crucial part of the pyrimidine biosynthesis pathway which generates dCTP and dTTP for incorporation into DNA. This reaction is required for DNA replication and cell growth. Thymidylate synthase activity is therefore required by all rapidly dividing cells such as cancer cells. Due to its association with DNA synthesis, and therefore, cellular replication, thymidylate synthase has been the target for anti-cancer drugs for many years. Non-limiting examples of thymidylate synthase inhibitors include folate and dUMP analogs, such as 5-fluorouracil (5-FU). Any drug that inhibits the expression of thymidylate synthase may be considered herein as a co-drug.

If desired, the administration of the nucleic acid composition described herein may be combined with one or more non-drug therapies, such as, for example, radiotherapy, and/or surgery. As well known in the art, radiation therapy and/or administration of the chemotherapeutic agent (in this case, the nucleic acid composition described herein, and optionally, any additional chemotherapeutic agent) may be given before surgery to, for example, shrink a tumor or stop the spread of the cancer before the surgery. As also well known in the art, radiation therapy and/or administration of the chemotherapeutic agent may be given after surgery to destroy any remaining cancer.

Examples have been set forth below for the purpose of illustration and to describe certain specific embodiments of the invention. However, the scope of this invention is not to be in any way limited by the examples set forth herein.

### EXAMPLES

### Example 1. Materials and Methods

Modified microRNAs. All modified microRNAs were synthesized by an automated oligonucleotide synthesis process and purified by HPLC. The two strands were annealed to make the mature modified 5-FU-miRs and/or modified miR-194 having cytosine bases replaced by a gemcitabine molecule of the present disclosure. For modified microRNA 194 containing a 5 halouracil, a process referred to as “2′-ACE RNA synthesis” was used. The 2′-ACE RNA synthesis is based on a protecting group scheme in which a silylether is employed to protect the 5′-hydroxyl group in combination with an acid-labile orthoester protecting group on the 2′-hydroxy (2′-ACE). This combination of protecting groups is then used with standard phosphoramidite solid-phase synthesis technology. See, for example, S. A. Scaringe, F. E. Wincott, and M. H. Caruthers, J. Am. Chem. Soc., 120 (45), 11820-11821 (1998); International PCT Application WO/1996/041809; M. D. Matteucci, M. H. Caruthers, J. Am. Chem. Soc., 103, 3185-3191 (1981); S. L. Beaucage, M. H. Caruthers, Tetrahedron Lett. 22, 1859-1862 (1981), the entire contents of each of which are expressly incorporated herein. The exemplary modified miR-194 nucleic acid or any other modified microRNAs that replace uracil with a 5-halouracil can be synthesized in the same manner as set forth herein.

Some exemplary structures of the protected and functionalized ribonucleoside phosphoramidites currently in use are shown below:

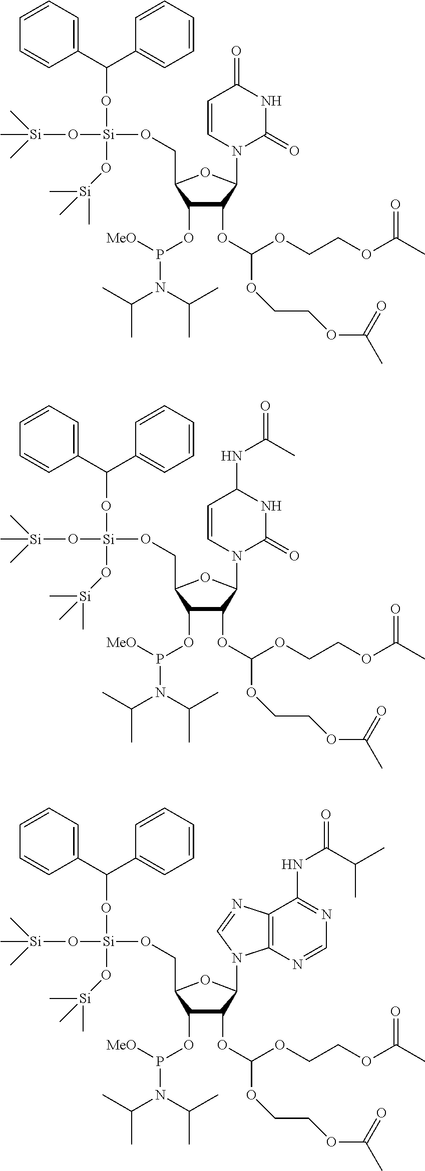

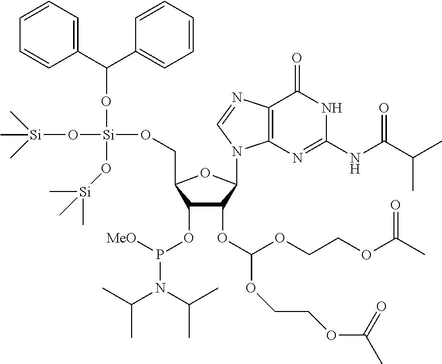

Modified miRs containing incorporating gemcitabine into miR-194 by replacing cytosine residues in its guide strand with gemcitabine (2′,2′-difluoro 2′-deoxycytidine) are synthesized as follows. 5′-Dimethoxytrityl-N₄-benzoyl-2′,2′-difluoro-2′-deoxycytidine (1 equiv., 0.4 mM, 270 mg) was dissolved in anhydrous acetonitrile (6 ml). A 0.5 M solution of ethylothiotetrazole (1.6 equiv., 0.65 mM, 1.3 ml) in anhydrous acetonitrile and 2-cyanoethyl-bis-(N, N′-diisopropropyl)phosphoramidite (1.43 equiv., 0.57 mmol, 0.2 ml) were added, and the reaction mixture stirred at room temperature for 1 h. The progress of the reaction was monitored by TLC and ³¹P NMR. When 5′-dimethoxytrityl-N₄-benzoyl-2′,2′-difluoro-2′-deoxycytidine was consumed in approximately 2 hours, the mixture was applied to a silica gel column, and the product eluted with 20% hexane in CH₂Cl₂, followed by MeOH in CHCl₃ (0-5% gradient). The desired product was identified by ³¹P NMR (CDCl₃): S 154.1, 152.1. The synthesis of all RNA oligonucleotides, unmodified and containing Gemcitabine units, was performed according to the phosphoramidite approach on a Gene World DNA synthesizer as set forth in K. Sipa, et al., RNA (2007) 13, pp. 1301-1316, the entire contents of which is incorporated herein by reference. The synthesis was carried out on a 200 nmol scale using appropriately protected phosphoramidite derivatives of thymidine, cytidine, uridine, guanosine, adenosine and 2′,2′-difluoro-2′-deoxycytidine, LCA-CPG as a solid support and 5-benzylmercaptotetrazole in anhydrous acetonitrile (0.25 M) as an activator. The synthesis had a prolonged coupling time (up to 600 seconds) for the modified unit. The coupling efficiency was determined by the DMT-cation assay.

Cell culture. The human pancreatic cancer cell lines ASPC-1, HS766T, and Panc-1, were obtained from the American Type Culture Collection (ATCC) and maintained in various types of media. Specifically, HS766T and PANC1 cells were cultured in DMEM containing media, and APSC-1 cells were maintained in RPMI medium (Thermo Fischer). Media was supplemented with 10% fetal bovine serum (Thermo Fischer).

Western immunoblot analysis. Twenty-four hours prior to transfection 1×10⁵ cells were plated in 6 well plates. Cells were either transfected using Oligofectamine (Thermo Fischer) or no transfection vehicle, with 50 nM control miRNA (Thermo Fischer), miR-194 or one of the three miR-194 mimics. Three days following transfection protein was collected in RIPA buffer with protease inhibitor (Sigma). Equal amounts of protein (15 μg), were separated on 10% sodium dodecyl sulfate-polyacrylamide gels as described in Laemmli U K. Nature. 1970; 227(5259) pp. 680-685, the entire contents of which is incorporated herein by reference. Proteins were probed with anti-SET8 or BMI-1 (1:500) (Cell Signaling Technologies) and anti-GAPDH (1:100000) antibodies. Horseradish peroxidase conjugated antibodies against mouse or rabbit (1:5000, Santa Cruz Biotech Inc.) were used as the secondary antibodies. Protein bands were visualized with autoradiography film using SuperSignal West Pico Chemiluminescent Substrate (Thermo Fischer).

Cell viability assay. Cells were plated 1000 cells per well in 96 well plates. Twenty four hours later cell media was changed to media supplemented with DFBS, with 50, 25, 12.5, 6.25, 3.125 or 1.5625 nM of Control miRNA (Thermo Fischer), miR-194, 5-FU-miR-194, Gem-mIR-194 or 5-FU-Gem-miR-194. 24 hours later media was changed again to fresh media supplemented with DFBS. 6 days after treatment cell number was assessed using WST-1 dye (Roche). Cells were incubated with 10 μl of WST-1 dye per 100 μl of media for 1 hour and absorbance was read at 450 and 630 nm. The optical density (O.D.) was calculated by subtracting the absorbance at 630 nm from that at 450 nm. IC⁵⁰ values were calculated using CompuSyn software (ComboSyn, Inc).

Mouse subcutaneous tumor implantation model. For in vivo miRNA delivery experiments, pancreatic cancer cells were created that expressed the lenti-luc reporter gene by infecting parental pancreatic cancer cells with a recombinant lentivirus. Luciferase-expressing HS766T cells (2.0×10⁶ cells per mouse) were suspended in 0.1 mL of PBS solution and was injected through tail vein of each mouse. Four days after injection of pancreatic cancer cells, mice were treated via tail vein injection with 80 μg of negative control or modified miR(s) packaged with in vivo-jet PEI (Polyplus Transfection). Mice were treated every other day for 2 weeks (8 times). Following treatment, mice were screened using IVIS Spectrum In vivo Imaging System (IVIS) (PerkinElmer).

Statistical analysis. All experiments were repeated at least three times. All statistical analyses were performed with SigmaPlot software. The statistical significance between two groups was determined using Student's t-test (paired t-test for clinical samples, and unpaired f-test for all other samples). For comparison of more than two groups, one-way ANOVA followed by a Bonferroni-Dunn test was used. Data were expressed as mean f standard error of the mean (SEM). The statistical significance is either described in figure legends, or indicated with asterisks (*). *=P<0.05; **=P<0.01; ***=P<0.001.

### Example 2: Modified miR-194 Nucleic Acids have Anti-Cancer Activity

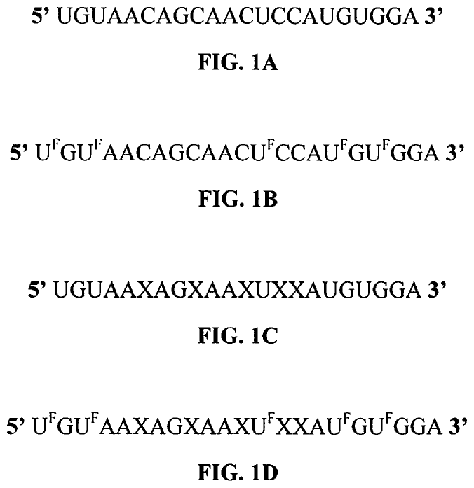

In the following experiments, all 5 cytosine bases in the guide strand of native miR-194 (SEQ ID NO:1) were replaced by gemcitabine to form the exemplary modified microRNA set forth in SEQ ID NO:2. See FIG. 1C. Another modified microRNA was formed by replacing all uracil bases in the guide strand of the native miR-194 nucleic acid with a 5-FU molecule, as set forth in SEQ ID NO. 4. See FIG. 1B. In one experiment, all U bases in miR-194 were replaced with 5-FU and all cytosine (C bases) were replaced by a gemcitabine molecule, as shown in the structure provided in FIG. 1D and as set forth in SEQ ID NO: 3.

Analysis of target specificity: The results of Western immunoblot experiments in pancreatic cells demonstrate that the exemplary modified miR-194 polynucleotides of the present disclosure were able to retain their target specificity to SET8 and BMI1. The results are shown in FIGS. 2A through 2D, which shows the results for the exemplary modified miR-194 nucleic acid having all U bases replaced with 5-FU, as set forth in SEQ ID. NO: 4 (5-FU-miR-194); the exemplary modified miR-194 nucleic acid having all U bases replaced with 5-FU, and all C bases replaced with gemcitabine as set forth in SEQ ID. NO: 3 (5-FU-Gem-miR-194); and the exemplary modified miR-194 nucleic acid having all C bases replaced with a gemcitabine molecule, as set forth in SEQ ID. NO: 2 (Gem-miR-194). Of further significance, the exemplary modified miR-194 nucleic acids were found to be more potent than unmodified (control) miR-194 in reducing the expression levels of SET8, as shown in FIG. 2A; and BMI1 as shown in FIG. 2C. When no transfection vehicle was used each of above modified miR-194 molecules retain the ability to inhibit SET8 (FIG. 2B) and BMI1 expression (FIG. 2D). This data demonstrates that both the 5-FU modification and the gemcitabine modification allow a modified miR-194 to enter the cell without any transfection vehicle and these modifications do not disrupt the ability of miR-194 to regulate target expression.

Exemplary modified microRNAs of the present disclosure inhibit tumor development and cell viability. The effects of each modified miR-194 molecule was tested in three different pancreatic cancer cell lines, ASPC1, PANC1 and HS766T and the results of such experiments are shown in FIGS. 3A-3C. As shown in FIG. 3A, when exogenously expressed in ASPC1 cells, all three modified miR-194 mimics exhibit efficacy in inhibiting cell viability when compared to exogenously expressed native miR-194. In ASPC1 cells, the IC50 for the exemplary modified miR-194 nucleic acid having all U bases replaced with 5-FU, as set forth in SEQ ID. NO: 4 (5-FU-miR-194) was 6.06 nM. The IC50 for the exemplary modified miR-194 nucleic acid having all C bases replaced with a gemcitabine molecule, as set forth in SEQ ID. NO: 2 (Gem-miR-194) was 4.29 nM and the IC50 for the exemplary modified miR-194 nucleic acid having all U bases replaced with 5-FU, and all C bases replaced with gemcitabine as set forth in SEQ ID. NO: 3 (5-FU-Gem-miR-194) was 2.88 nM (Table 1).

As shown in FIG. 3B, when exogenously expressed in PANC1 cells, there was a clear difference in the effects of the exemplary modified miR-194 nucleic acid having all U bases replaced with 5-FU (5-FU-miR-194) compared to both the exemplary modified miR-194 nucleic acid having all C bases replaced with a gemcitabine molecule, as set forth in SEQ ID. NO: 2 (Gem-miR-194) and the exemplary modified miR-194 nucleic acid having all U bases replaced with 5-FU, and all C bases replaced with gemcitabine as set forth in SEQ ID. NO: 3 (5-FU-Gem-miR-194). Specifically, the IC50 for 5-FU-miR-194 was 16 nM, while those for Gem-miR-194 and the 5-FU-Gem-miR-194 were 1.92 and 0.93 respectively (Table 1).

In addition and as shown in FIG. 3C, when exogenously expressed in HS766T cells, the greatest difference between the 5-FU modified miR-194 and both the exemplary modified miR-194 nucleic acid having all C bases replaced with a gemcitabine molecule, as set forth in SEQ ID. NO: 2 (Gem-miR-194) and the exemplary modified miR-194 nucleic acid having all U bases replaced with 5-FU, and all C bases replaced with gemcitabine as set forth in SEQ ID. NO: 3 (5-FU-Gem-miR-194) was observed. Here, the IC50 for 5-FU-miR-194 was 26.45 nM while the IC50 for Gem-mir-194 and the 5-FU-Gem-miR-194 were 3.57 and 2.46 nM respectively (Table 1).

TABLE 1
IC50 for each exemplary modified miR-194 nucleic acid
in 3 different pancreatic cancer cell lines (nM).

| Treatment | ASPC1 | PANC1 | HS766T |
|---|---|---|---|
| miR-194 | — | — | — |
| 5-FU-miR-194 | 6.06 | 16.71 | 26.45 |
| Gem-miR-194 | 4.29 | 1.92 | 3.57 |
| 5-FU-Gem-miR-194 | 2.88 | 0.93 | 2.46 |

Taken together, these data show that the incorporation of gemcitabine into miR-194 enhances its effects on inhibiting the viability of pancreatic cancer cells when compared to microRNAs modified by replacing U-bases with 5-FU alone. Furthermore, the data reveals that miR-194 having all U bases replaced with 5-FU, and all C bases replaced with gemcitabine as set forth in SEQ ID. NO: 3 (5-FU-Gem-miR-194) has the greatest efficacy in treating on pancreatic cancer.

Modified miR-194 inhibits cancer growth in vivo. A mouse xenograft model was established that included pancreatic cancer cells as shown in FIG. 4. Four days after establishing metastasis, 80 μg of modified miR-194 nucleic acid composition (SEQ ID NO: 2, Gem-miR-194) or negative control microRNA (Control) was delivered by intravenous injection with a treatment frequency of one injection every other day for two weeks. The exemplary modified miR-194 nucleic acid was able to inhibit metastatic pancreatic cancer growth compared to control as shown in FIG. 4. Furthermore, mice treated with modified miR-194 nucleic acids did not exhibit any toxicity.

The data presented here supports the viability of a novel modification in which gemcitabine is incorporated into a miRNA nucleic acid sequence to enhance the chemotherapeutic function of the native microRNA molecule with or without the use of other chemotherapeutic agents.

1. A nucleic acid composition comprising a modified microRNA nucleotide sequence that comprises at least one cytosine nucleic acid, wherein one or more of said at least one cytosine nucleic acids is replaced by a gemcitabine molecule.

2. The nucleic acid composition of claim 1, wherein at least two of the cytosine nucleic acids in the nucleotide sequence are each replaced by a gemcitabine molecule.

3. The nucleic acid composition of claim 2, wherein at least three of the cytosine nucleic acids in the nucleotide sequence are each replaced by a gemcitabine molecule.

4.-5. (canceled)

6. The nucleic acid composition of claim 1, wherein said modified microRNA nucleotide sequence comprises a microRNA nucleotide sequence of miR-194.

7. The nucleic acid composition of claim 6, wherein said modified miR-194 comprises the sequence set forth in SEQ ID NO: 2.

8. The nucleic acid composition of claim 6, wherein said modified miR-194 comprises the sequence set forth in SEQ ID NO: 4.

9. A nucleic acid composition comprising a modified microRNA nucleotide sequence that comprises at least one cytosine nucleic acid and at least one uracil nucleic acid, wherein one or more of said at least one cytosine nucleic acids is replaced by a gemcitabine molecule and one or more of said at least one uracil nucleic acids is replaced by a 5-halouracil.

10. The nucleic acid composition of claim 9, wherein at least two of the cytosine nucleic acids in the nucleotide sequence are each replaced by a gemcitabine molecule.

11. The nucleic acid composition of claim 10, wherein at least two of the uracil nucleic acids are each replaced by a 5-halouracil.

12. The nucleic acid composition of claim 9, wherein all of the cytosine nucleic acids in the nucleotide sequence are replaced by a gemcitabine molecule.

13. The nucleic acid composition of claim 12, wherein all of the uracil nucleic acids are each replaced by a 5-halouracil.

14. The nucleic acid composition of claim 9, wherein said modified microRNA nucleotide sequence comprises a microRNA nucleotide sequence of miR-194.

15. The nucleic acid composition of claim 14, wherein said modified miR-194 comprises the sequence set forth in SEQ ID NO: 2.

16. The nucleic acid composition of claim 14, wherein said modified miR-194 comprises the sequence set forth in SEQ ID NO: 4.

17. The nucleic acid composition of claim 9, wherein said 5-halouracil is 5-fluorouracil.

18. A pharmaceutical composition comprising at least one nucleic acid composition of claim 1.

19. The pharmaceutical composition of claim 18, wherein the nucleic acid composition comprises a modified microRNA nucleotide sequence selected from the group consisting of SEQ ID NO: 2 and SEQ ID NO: 4.

20. A method for treating cancer comprising administering to a subject an effective amount of a nucleic acid composition of claim 1, wherein said subject has cancer, and wherein progression of said cancer is inhibited.

21. (canceled)

22. The method of claim 20, wherein said subject has a cancer selected from the group consisting of, pancreatic cancer, bladder cancer, lung cancer and ovarian cancer.

23. (canceled)

24. The method of claim 20, wherein the nucleic acid composition comprises a modified microRNA nucleotide sequence selected from the group consisting of SEQ ID NO: 2, SEQ ID NO: 4 and combinations thereof.

25.-26. (canceled)
